(19) United States
(12) Patent Application Publication
(10) Pub. No.: US 2010/0015231 A1
(43) Pub. Date: Jan. 21, 2010

(54) LOW SWELL, LONG-LIVED HYDROGEL SEALANT

(75) Inventor: Helen S. M. LU, Wallingford, PA (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E.I. DU PONT DE NEMOURS AND COMPANY, Wilmington, DE (US)

(21) Appl. No.: 12/497,729

(22) Filed: Jul. 6, 2009

Related U.S. Application Data
(60) Provisional application No. 61/135,172, filed on Jul. 17, 2008.

(51) Int. Cl. A61K 9/10

(52) U.S. Cl. 424/488

(57) ABSTRACT

A low swell, long-lived hydrogel sealant formed by reacting a highly oxidized polysaccharide containing aldehyde groups with a multi-arm amine is described. The hydrogel sealant may be particularly suitable for applications requiring low swell and slow degradation, for example, ophthalmic applications such as sealing wounds resulting from trauma such as corneal lacerations, or from surgical procedures such as vitrectomy procedures, cataract surgery, LASIK surgery, glaucoma surgery, and corneal transplants; neurosurgery applications, such as sealing the dura; and as a plug to seal a fistula or the punctum. The low swell, long-lived hydrogel sealant may also be useful as a tissue sealant and adhesive, and as an anti-adhesion barrier.

### CROSS-REFERENCE TO RELATED APPLICATION

This application claims priority under 35 U.S.C. §119 from U.S. Provisional Application Ser. No. 61/135,172, filed Jul. 17, 2008.

### FIELD OF THE INVENTION

The invention relates to the field of medical adhesives. More specifically, the invention relates to a low swell, long-lived hydrogel sealant formed by reacting a highly oxidized polysaccharide containing aldehyde groups with a multi-arm amine.

### BACKGROUND OF THE INVENTION

Tissue adhesives have many potential medical applications, including wound closure, supplementing or replacing sutures or staples in internal surgical procedures, adhesion of synthetic onlays or inlays to the cornea, drug delivery devices, and as anti-adhesion barriers to prevent post-surgical adhesions. Conventional tissue adhesives are generally not suitable for a wide range of adhesive applications. For example, cyanoacrylate-based adhesives have been used for topical wound closure, but the release of toxic degradation products limits their use for internal applications. Fibrin-based adhesives are slow curing, have poor mechanical strength, and pose a risk of viral infection. Additionally, the fibrin-based adhesives do not bond covalently to the underlying tissue.

Several types of hydrogel tissue adhesives have been developed, which have improved adhesive and cohesive properties and are nontoxic. These hydrogels are generally formed by reacting a component having nucleophilic groups with a component having electrophilic groups, which are capable of reacting with the nucleophilic groups of the first component, to form a crosslinked network via covalent bonding. However, these hydrogels typically swell or dissolve away too quickly, or lack sufficient adhesion or mechanical strength, thereby decreasing their effectiveness as surgical adhesives.

Kodokian et al. (copending and commonly owned U.S. Patent Application Publication No. 2006/0078536) describe hydrogel tissue adhesives formed by reacting an oxidized polysaccharide with a water-dispersible, multi-arm polyether amine. These adhesives provide improved adhesion and cohesion properties, crosslink readily at body temperature, maintain dimensional stability initially, do not degrade rapidly, and are nontoxic to cells and non-inflammatory to tissue. However, for certain applications, for example, ophthalmic applications such as sealing wounds resulting from trauma such as corneal lacerations, or from surgical procedures such as vitrectomy procedures, cataract surgery, LASIK surgery, glaucoma surgery, and corneal transplants; neurosurgery applications, such as sealing the dura; and as a plug to seal a fistula or the punctum, hydrogel tissue adhesives with low swell and slow degradation are needed.

Lu (copending and commonly owned Patent Application No. PCT/US08/83545) describes a dextran-based hydrogel sealant formed by reacting an aminodextran containing primary amine groups with an oxidized dextran containing aldehyde groups which has low swell and a slow degradation rate. However, an adhesive with even slower degradation at comparable solid levels would be beneficial for the applications listed above.

Therefore, the problem to be solved is to provide a hydrogel material with low swell and a slow degradation rate for use in certain surgical procedures and other medical applications that require these properties.

### SUMMARY OF THE INVENTION

The stated problem is addressed herein by the discovery that a low swell, long-lived hydrogel sealant is formed by reacting at least one highly oxidized polysaccharide containing aldehyde groups with at least one water-dispersible, multi-arm amine at the conditions described herein. Methods of using the low swell, long-lived hydrogel sealant for medical purposes are also provided.

Accordingly, in one aspect the present invention provides a kit comprising:
a) a first aqueous solution or dispersion comprising at least one highly oxidized polysaccharide containing aldehyde groups, said highly oxidized polysaccharide having a weight-average molecular weight of about 1,000 to about 1,000,000 Daltons and an equivalent weight per aldehyde group of about 65 to about 85 Daltons, wherein said first aqueous solution or dispersion contains said highly oxidized polysaccharide at a concentration of 6% to about 40% by weight; and
b) a second aqueous solution or dispersion comprising at least one water-dispersible, multi-arm amine wherein at least three of the arms are terminated by at least one primary amine group, said multi-arm amine having a number-average molecular weight of about 450 to about 200,000 Daltons, wherein said second aqueous solution or dispersion contains said multi-arm amine at a concentration of about 5% to about 70% by weight;
   provided that:
(i) if the concentration of the highly oxidized polysaccharide in the first aqueous solution or dispersion is equal to or greater than 6 wt % but less than 8 wt %, the equivalent weight per aldehyde group of the highly oxidized polysaccharide is about 65 to about 70 Daltons;
(ii) if the concentration of the highly oxidized polysaccharide in the first aqueous solution or dispersion is equal to or greater than 8 wt % to about 40 wt %, the equivalent weight per aldehyde group of the highly oxidized polysaccharide is about 65 to about 85 Daltons.

In another embodiment, the invention provides a dried hydrogel formed by a process comprising the steps of:
a) combining (i) a first solution or dispersion comprising at least one highly oxidized polysaccharide containing aldehyde groups in a first solvent, said oxidized polysaccharide having a weight-average molecular weight of about 1,000 to about 1,000,000 Daltons and an equivalent weight per aldehyde group of about 65 to about 85 Daltons, wherein said first solution or dispersion contains said highly oxidized polysaccharide at a concentration of 6% to about 40% by weight; with (ii) a second solution or dispersion comprising at least one water-dispersible, multi-arm amine in a second solvent, wherein at least three of the arms of said multi-arm amine are terminated by at least one primary amine group, said multi-arm amine having a number-average molecular weight of about 450 to about 200,000 Daltons, wherein said second solution or dispersion contains said multi-arm amine at a concentration of about 5% to about 70% by weight, to form a hydrogel, wherein the first solvent is either the same as or different from the second solvent; and
b) treating the hydrogel to remove at least a portion of said first solvent and said second solvent to form the dried hydrogel;
provided that:
(i) if the concentration of the highly oxidized polysaccharide in the first solution or dispersion is equal to or greater than 6 wt % but less than 8 wt %, the equivalent weight per aldehyde group of the highly oxidized polysaccharide is about 65 to about 70 Daltons;
(ii) if the concentration of the highly oxidized polysaccharide in the first solution or dispersion is equal to or greater than 8 wt % to about 40 wt %, the equivalent weight per aldehyde group of the highly oxidized polysaccharide is about 65 to about 85 Daltons.

In another aspect, the present invention provides method for applying a coating to an anatomical site on tissue of a living organism comprising: applying to the site (a) a first aqueous solution or dispersion comprising at least one highly oxidized polysaccharide containing aldehyde groups, said highly oxidized polysaccharide having a weight-average molecular weight of about 1,000 to about 1,000,000 Daltons and an equivalent weight per aldehyde group of about 65 to about 85 Daltons, wherein said first aqueous solution or dispersion contains said highly oxidized polysaccharide at a concentration of 6% to about 40% by weight; followed by (b) a second aqueous solution or dispersion comprising at least one water-dispersible, multi-arm amine wherein at least three of the arms are terminated by at least one primary amine group, said multi-arm amine having a number-average molecular weight of about 450 to about 200,000 Daltons, wherein said second aqueous solution or dispersion contains said multi-arm amine at a concentration of about 5% to about 70% by weight, or applying (b) followed by (a) and mixing (a) and (b) on the site, or

premixing (a) and (b) to form a mixture and applying said mixture to the site;

provided that:
(i) if the concentration of the highly oxidized polysaccharide in the first aqueous solution or dispersion is equal to or greater than 6 wt % but less than 8 wt %, the equivalent weight per aldehyde group of the highly oxidized polysaccharide is about 65 to about 70 Daltons;
(ii) if the concentration of the highly oxidized polysaccharide in the first aqueous solution or dispersion is equal to or greater than 8 wt % to about 40 wt %, the equivalent weight per aldehyde group of the highly oxidized polysaccharide is about 65 to about 85 Daltons.

In another aspect, the invention provides a method for bonding at least two anatomical sites together comprising: applying to at least one site (a) a first aqueous solution or dispersion comprising at least one highly oxidized polysaccharide containing aldehyde groups, said highly oxidized polysaccharide having a weight-average molecular weight of about 1,000 to about 1,000,000 Daltons and an equivalent weight per aldehyde group of about 65 to about 85 Daltons, wherein said first aqueous solution or dispersion contains said highly oxidized polysaccharide at a concentration of 6% to about 40% by weight; applying to at least one of either the same site or one other site (b) a second aqueous solution or dispersion comprising at least one water-dispersible, multi-arm amine wherein at least three of the arms are terminated by at least one primary amine group, said multi-arm amine having a number-average molecular weight of about 450 to about 200,000 Daltons, wherein said second aqueous solution or dispersion contains said multi-arm amine at a concentration of about 5% to about 70% by weight; or premixing (a) and (b) to form a mixture and applying said mixture to at least one site before the mixture completely cures; and contacting the at least two anatomical sites together;

provided that:
(i) if the concentration of the highly oxidized polysaccharide in the first aqueous solution or dispersion is equal to or greater than 6 wt % but less than 8 wt %, the equivalent weight per aldehyde group of the highly oxidized polysaccharide is about 65 to about 70 Daltons;
(ii) if the concentration of the highly oxidized polysaccharide in the first aqueous solution or dispersion is equal to or greater than 8 wt % to about 40 wt %, the equivalent weight per aldehyde group of the highly oxidized polysaccharide is about 65 to about 85 Daltons.

In another aspect, the present invention provides a composition comprising the reaction product of:
a) a first aqueous solution or dispersion comprising at least one highly oxidized polysaccharide containing aldehyde groups, said highly oxidized polysaccharide having a weight-average molecular weight of about 1,000 to about 1,000,000 Daltons and an equivalent weight per aldehyde group of about 65 to about 85 Daltons, wherein said first aqueous solution or dispersion contains said highly oxidized polysaccharide at a concentration of 6% to about 40% by weight; and
b) a second aqueous solution or dispersion comprising at least one water-dispersible, multi-arm amine wherein at least three of the arms are terminated by at least one primary amine group, said multi-arm amine having a number-average molecular weight of about 450 to about 200,000 Daltons, wherein said second aqueous solution or dispersion contains said multi-arm amine at a concentration of about 5% to about 70% by weight;
   provided that:
(i) if the concentration of the highly oxidized polysaccharide in the first aqueous solution or dispersion is equal to or greater than 6 wt % but less than 8 wt %, the equivalent weight per aldehyde group of the highly oxidized polysaccharide is about 65 to about 70 Daltons;
(ii) if the concentration of the highly oxidized polysaccharide in the first aqueous solution or dispersion is equal to or greater than 8 wt % to about 40 wt %, the equivalent weight per aldehyde group of the highly oxidized polysaccharide is about 65 to about 85 Daltons.

### DETAILED DESCRIPTION OF THE INVENTION

Disclosed herein is a low swell, long-lived hydrogel sealant formed by reacting at least one highly oxidized polysaccharide containing aldehyde groups, having an equivalent weight per aldehyde group of about 65 to about 85 Daltons, with at least one water-dispersible, multi-arm amine at the conditions disclosed herein. The low swell, long-lived hydrogel sealant may be particularly useful in medical applications where low swell and slow degradation are needed, including but not limited to, ophthalmic applications such as sealing wounds resulting from trauma such as corneal lacerations, or from surgical procedures such as vitrectomy procedures, cataract surgery, LASIK surgery, glaucoma surgery, and corneal transplants; neurosurgery applications, such as sealing the dura; and as a plug to seal a fistula or the punctum. The low swell, long-lived hydrogel sealant may also be useful as a tissue sealant and adhesive, and as an anti-adhesion barrier.

The following definitions are used herein and should be referred to for interpretation of the claims and the specification.

The term “oxidized polysaccharide” refers to a polysaccharide that has been reacted with an oxidizing agent to introduce aldehyde groups into the molecule.

The term “highly oxidized polysaccharide” as used herein, refers to an oxidized polysaccharide that has an equivalent weight per aldehyde group of about 65 to about 85 Daltons.

The term “equivalent weight per aldehyde group” refers to the average molecular weight of the compound divided by the number of aldehyde groups in the molecule.

The term “water-dispersible, multi-arm amine” refers to a polymer having three or more polymer chains (“arms”), which may be linear or branched, emanating from a central structure, which may be a single atom, a core molecule, or a polymer backbone, wherein at least three of the branches (“arms”) are terminated by at least one primary amine group. The water-dispersible, multi-arm amine is water soluble or is able to be dispersed in water to form a colloidal suspension capable of reacting with a second reactant in aqueous solution or dispersion.

The term “dispersion” as used herein, refers to a colloidal suspension capable of reacting with a second reactant in an aqueous medium.

The term “water-dispersible, multi-arm polyether amine” refers to a water-dispersible, multi-arm amine wherein the polymer is a polyether.

The term “polyether” refers to a polymer having the repeat unit [—O—R]—, wherein R is a hydrocarbylene group having 2 to 5 carbon atoms. The polyether may also be a random or block copolymer comprising different repeat units which contain different R groups.

The term “hydrocarbylene group” refers to a divalent group formed by removing two hydrogen atoms, one from each of two different carbon atoms, from a hydrocarbon.

The term “branched polyether” refers to a polyether having one or more branch points (“arms”), including star, dendritic, comb, highly branched, and hyperbranched polyethers.

The term “dendritic polyether” refers to a highly branched polyether having a branching structure that repeats regularly with each successive generation of monomer, radiating from a core molecule.

The term “comb polyether” refers to a multi-arm polyether in which linear side chains emanate from trifunctional branch points on a linear polymer backbone.

The term “star polyether” refers to a multi-arm polyether in which linear side chains emanate from a single atom or a core molecule having a point of symmetry.

The term “highly branched polyether” refers to a multi-arm polyether having many branch points, such that the distance between branch points is small relative to the total length of the arms.

The term “hyperbranched polyether” refers to a multi-arm polyether that is more branched than highly branched with order approaching that of an imperfect dendritic polyether.

The term “branched end amine” refers to a linear or multi-arm polymer having two or three primary amine groups at each of the ends of the polymer chain or at the end of the polymer arms.

The term “multi-functional amine” refers to a chemical compound comprising at least two functional groups, at least one of which is a primary amine group.

The term “hydrogel” refers to a water-swellable polymeric matrix, consisting of a three-dimensional network of macromolecules held together by covalent or non-covalent crosslinks, that can absorb a substantial amount of water to form an elastic gel.

The term “low swell, long-lived hydrogel” as used herein, refers to a hydrogel formed by reacting a highly oxidized polysaccharide having an equivalent weight per aldehyde group of about 65 to about 85 Daltons and a multi-arm amine at the conditions described herein. The low swell, long-lived hydrogel has a lower maximum swell and a slower degradation than a hydrogel formed from the same oxidized polysaccharide having an equivalent weight per aldehyde group of greater than or equal to about 90 Daltons and the same multi-arm amine at the same total solids content.

The term “maximum swell” as used herein, refers to the maximum weight that a hydrogel attains when soaked in an aqueous solution, such as phosphate buffered saline (PBS), for a period of time, divided by the initial weight of the hydrogel, multiplied by 100.

The term “dried hydrogel” refers to a hydrogel that has been treated to remove at least a portion of the solvent(s) contained therein. Preferably, substantially all of the solvent(s) is/are removed from the hydrogel.

The term “% by weight”, also referred to herein as “wt %”, refers to the weight percent relative to the total weight of the solution or dispersion, unless otherwise specified.

The term “anatomical site” refers to any external or internal part of the body of humans or animals.

The term “tissue” refers to any tissue, both living and dead, in humans or animals.

By medical application is meant medical applications as related to humans and animals.

### Highly Oxidized Polysaccharides

The polysaccharides useful in the invention are oxidized to contain aldehyde groups. Suitable starting polysaccharides include, but are not limited to, dextran, starch, agar, cellulose, and hyaluronic acid. These polysaccharides are available commercially from sources such as Sigma-Aldrich (Milwaukee, Wis.) and Pharmacosmos A/S (Holbaek, Denmark). Typically, commercial preparations of polysaccharides are a heterogeneous mixture having a distribution of different molecular weights and are characterized by various molecular weight averages, for example, the weight-average molecular weight, or the number-average molecular weight, as is known in the art. Suitable polysaccharides have a weight-average molecular weight of about 1,000 to about 1,000,000 Daltons, more particularly from about 3,000 to about 500,000 Daltons.

The polysaccharides may be oxidized to contain aldehyde groups using methods known in the art. Highly oxidized polysaccharides may be prepared by oxidation of polysaccharides using any suitable oxidizing agent, including but not limited to, periodates, hypochlorites, ozone, peroxides, hydroperoxides, persulfates, and percarbonates. In one embodiment, the polysaccharide is oxidized by reaction with sodium periodate, for example as described by Mo et al. (J. Biomater. Sci. Polymer Edn. 11:341-351, 2000). Additionally, the highly oxidized polysaccharide may be prepared using the method described by Cohen et al. (copending and commonly owned Patent Application No. PCT/US08/05013 (WO 2008/133847)). That method of making an oxidized polysaccharide comprises a combination of precipitation and separation steps to purify the oxidized polysaccharide formed by oxidation of the polysaccharide with periodate and provides an oxidized polysaccharide with very low levels of iodine-containing species. The polysaccharide may be reacted with different amounts of periodate to give polysaccharides with different degrees of oxidation and therefore, different amounts of aldehyde groups, as described in detail in the General Methods section of the Examples herein. Specifically, the amount of oxidizing agent is chosen to provide a highly oxidized polysaccharide having an equivalent weight per aldehyde group of about 65 to about 85 Daltons.

The aldehyde content of the highly oxidized polysaccharide may be determined using methods known in the art. For example, the dialdehyde content of the highly oxidized polysaccharide, also referred to herein as the oxidation conversion, may be determined using the method described by Hofreiter et al. (Anal Chem. 27:1930-1931, 1955). In that method, the amount of alkali consumed per mole of dialdehyde in the highly oxidized polysaccharide, under specific reaction conditions, is determined by a pH titration. Alternatively, the oxidation conversion of the highly oxidized polysaccharide may be determined using nuclear magnetic resonance (NMR) spectroscopy, as described in the Examples herein.

Suitable highly oxidized polysaccharides containing aldehyde groups have a weight-average molecular weight of about 1,000 to about 1,000,000 Daltons, more particularly from about 3,000 to about 500,000 Daltons; and an equivalent weight per aldehyde group of about 65 to about 85 Daltons. In one embodiment, the equivalent weight per aldehyde group of the highly oxidized polysaccharide is about 70 to about 80 Daltons. In another embodiment, the equivalent weight per aldehyde group of the highly oxidized polysaccharide is about 65 to about 70 Daltons.

In one embodiment, the highly oxidized polysaccharide containing aldehyde groups is oxidized dextran having a weight-average molecular weight of about 8,500 to about 11,500 Daltons and an equivalent weight per aldehyde group of about 73 Daltons (oxidation conversion of about 91%).

In another embodiment, the highly oxidized polysaccharide containing aldehyde groups is oxidized dextran having a weight-average molecular weight of about 8,500 to about 11,500 Daltons and an equivalent weight per aldehyde group of about 80 Daltons (oxidation conversion of about 84%).

In another embodiment, the highly oxidized polysaccharide containing aldehyde groups is oxidized dextran having a weight-average molecular weight of about 60,000 to about 90,000 Daltons and an equivalent weight per aldehyde group of about 71 Daltons (oxidation conversion of about 93%).

### Water-Dispersible, Multi-Arm Amines

Suitable water dispersible, multi-arm amines include, but are not limited to, water dispersible multi-arm polyether amines, amino-terminated dendritic polyamidoamines, and multi-arm branched end amines. Typically, the multi-arm amines have a number-average molecular weight of about 450 to about 200,000 Daltons, more particularly from about 2,000 to about 40,000 Daltons.

In one embodiment, the water dispersible, multi-arm amine is a multi-arm polyether amine, which is a water-dispersible polyether having the repeat unit [—O—R]—, wherein R is a hydrocarbylene group having 2 to 5 carbon atoms. Suitable multi-arm polyether amines include, but are not limited to, dendritic, comb, star, highly branched, and hyperbranched polyethers wherein at least three of the arms are terminated by at least one primary amine group. Examples of water-dispersible, multi-arm polyether amines include, but are not limited to, amino-terminated star polyethylene oxides, amino-terminated dendritic polyethylene oxides, amino-terminated comb polyethylene oxides, amino-terminated star polypropylene oxides, amino-terminated dendritic polypropylene oxides, amino-terminated comb polypropylene oxides, amino-terminated star polyethylene oxide-polypropylene oxide copolymers, amino-terminated dendritic polyethylene oxide-polypropylene oxide copolymers, amino-terminated comb polyethylene oxide-polypropylene oxide copolymers, and polyoxyalkylene triamines sold under the trade name Jeffamine® triamines, by Huntsman LLC. (Houston, Tex.). Examples of star polyethylene oxide amines, include, but are not limited to, various multi-arm polyethylene glycol amines, available for example from Nektar Transforming Therapeutics (Huntsville, Ala.), and star polyethylene glycols having 3, 4, 6, or 8 arms terminated with primary amines (referred to herein as 3, 4, 6 or 8-arm star PEG amines, respectively). The 8-arm star PEG amine is available from Nektar Transforming Therapeutics. Examples of suitable Jeffamine® triamines include, but are not limited to, Jeffamine® T-403 (CAS No. 39423-51-3), Jeffamine® T-3000 (CAS No. 64852-22-8), and Jeffamine® T-5000 (CAS No. 64852-22-8).

In one embodiment, the water-dispersible multi-arm amine is an eight-arm polyethylene glycol having eight arms terminated by a primary amine group and having a number-average molecular weight of about 10,000 Daltons.

The multi-arm polyether amines are either available commercially, as noted above, or may be prepared using methods known in the art. For example, multi-arm polyethylene glycols, wherein at least three of the arms are terminated by a primary amine group, may be prepared by putting amine ends on multi-arm polyethylene glycols (e.g., 3, 4, 6, and 8-arm star polyethylene glycols, available from companies such as Nektar Transforming Therapeutics; SunBio, Inc., Anyang City, South Korea; NOF Corp., Tokyo, Japan; or JenKem Technology USA, Allen, Tex.) using the method described by Buckmann et al. (Makromol. Chem. 182:1379-1384, 1981). In that method, the multi-arm polyethylene glycol is reacted with thionyl bromide to convert the hydroxyl groups to bromines, which are then converted to amines by reaction with ammonia at 100° C. The method is broadly applicable to the preparation of other multi-arm polyether amines. Additionally, multi-arm polyether amines may be prepared from multi-arm polyols using the method described by Chenault (copending and commonly owned U.S. Patent Application Publication No. 2007/0249870). In that method, the multi-arm polyether is reacted with thionyl chloride to convert the hydroxyl groups to chlorine groups, which are then converted to amines by reaction with aqueous or anhydrous ammonia. Other methods that may used for preparing multi-arm polyether amines are described by Merrill et al. in U.S. Pat. No. 5,830,986, and by Chang et al. in WO 97/30103.

The multi-arm amine may also be an amino-terminated dendritic polyamidoamine, sold under the trade name Starburst® Dendrimers (available from Sigma-Aldrich, St Louis, Mo.).

The multi-arm amine may also be a multi-arm branched end amine, as described by Arthur (copending and commonly owned Patent Application No. PCT/US07/24393 (WO 2008/066787)). The multi-arm branched end amines are branched polymers having two or three primary amine groups at the end of each of the polymer arms. The multiplicity of functional groups increases the statistical probability of reaction at a given chain end and allows more efficient incorporation of the molecules into a polymer network. The starting materials used to prepare the multi-arm branched end amines are branched polymers such as multi-arm polyether polyols including, but not limited to, comb and star polyether polyols. The multi-arm branched end amines can be prepared by attaching multiple amine groups to the end of the polymer arms using methods well known in the art. For example, a multi-arm branched end amine having two primary amine functional groups on the end of each of the polymer arms can prepared by reacting the starting material, as listed above, with thionyl chloride in a suitable solvent such as toluene to give the chloride derivative, which is subsequently reacted with tris(2-aminoethyl)amine to give the multi-arm branched end reactant having two amine groups at the end of the polymer arms.

It should be recognized that the multi-arm amines are generally a somewhat heterogeneous mixture having a distribution of arm lengths and in some cases, a distribution of species with different numbers of arms. When a multi-arm amine has a distribution of species having different numbers of arms, it can be referred to based on the average number of arms in the distribution. For example, in one embodiment the multi-arm amine is an 8-arm star PEG amine, which comprises a mixture of multi-arm star PEG amines, some having less than and some having more than 8 arms; however, the multi-arm star PEG amines in the mixture have an average of 8 arms. Therefore, the terms “8-arm”, “6-arm”, “4-arm” and “3-arm” as used herein to refer to multi-arm amines, should be construed as referring to a heterogeneous mixture having a distribution of arm lengths and in some cases, a distribution of species with different numbers of arms, in which case the number of arms recited refers to the average number of arms in the mixture.

In one embodiment, the polysaccharide that is highly oxidized to contain aldehyde groups is dextran and the multi-arm amine is a multi-arm polyethylene glycol amine.

### Methods of Using the Low Swell, Long-Lived Hydrogel Sealant

The low swell, long-lived hydrogel sealant disclosed herein may be used in various forms. In one embodiment, the highly oxidized polysaccharide containing aldehyde groups and the multi-arm amine are used in the form of aqueous solutions or dispersions. To prepare an aqueous solution or dispersion comprising at least one highly oxidized polysaccharide (referred to herein as the “first aqueous solution or dispersion”), at least one highly oxidized polysaccharide, as described above, is added to water to give a concentration of 6% to about 40%, more particularly about 8% to about 20% by weight relative to the total weight of the solution or dispersion. Mixtures of different highly oxidized polysaccharides, having different average molecular weights and/or different equivalent weights per aldehyde group may also be used. If a mixture of different highly oxidized polysaccharides is used, the total concentration of the polysaccharides is 6% to about 40%, more particularly about 8% to about 20% by weight relative to the total weight of the solution or dispersion. In one embodiment, the concentration of the highly oxidized polysaccharide in the first aqueous solution or dispersion is about 8% by weight relative to the total weight of the solution or dispersion.

The degree of swell of the low swell, long-lived hydrogel sealant disclosed herein is governed by its crosslink density, which depends on several factors including the concentration of the highly oxidized polysaccharide in the first aqueous solution or dispersion and the equivalent weight per aldehyde group of the highly oxidized polysaccharide. Therefore, where the concentration of the highly oxidized polysaccharide in the first aqueous solution or dispersion is equal to or greater than 6 wt % but less than 8 wt %, the equivalent weight per aldehyde group of the highly oxidized polysaccharide is about 65 to about 70 Daltons in order to obtain a crosslink density that results in a hydrogel with low swell. Where the concentration of the highly oxidized polysaccharide in the first aqueous solution or dispersion is equal to or greater than 8 wt % to about 40 wt %, the equivalent weight per aldehyde group of the highly oxidized polysaccharide is about 65 to about 85 Daltons.

To prepare an aqueous solution or dispersion comprising at least one water-dispersible, multi-arm amine (referred to herein as the “second aqueous solution or dispersion”), at least one multi-arm amine is added to water to give a concentration of about 5% to about 70% by weight, more particularly about 15% to about 50% by weight, and more particularly about 30% to about 50% by weight, relative to the total weight of the solution or dispersion. Mixtures of different multi-arm amines may also be used. If a mixture of different multi-arm amines is used, the total concentration of the multi-arm amines is about 5% to about 70% by weight, more particularly about 15% to about 50% by weight, and more particularly about 30% to about 50% by weight, relative to the total weight of the solution or dispersion. The optimal concentrations of the two aqueous solutions or dispersions to be used depend on the application, and can be readily determined by one skilled in the art using routine experimentation.

For use on living tissue, it is preferred that the first aqueous solution or dispersion and the second aqueous solution or dispersion be sterilized to prevent infection. Any suitable sterilization method known in the art that does not adversely affect the ability of the components to react to form an effective hydrogel may be used, including, but not limited to, electron beam irradiation, gamma irradiation, ethylene oxide sterilization, or ultra-filtration through a 0.2 μm pore membrane.

The first aqueous solution or dispersion and/or the second aqueous solution or dispersion may further comprise various additives depending on the intended application. Preferably, the additive is compatible with the other components of the solution. Specifically, the additive does not contain groups that would interfere with effective gelation of the hydrogel. The amount of the additive used depends on the particular application and may be readily determined by one skilled in the art using routine experimentation. For example, the solution(s) or dispersion(s) may comprise at least one additive selected from the group consisting of pH modifiers, viscosity modifiers, colorants, surfactants, pharmaceutical drugs and therapeutic agents.

The solution(s) or dispersion(s) may optionally include at least one pH modifier to adjust the pH of the solution(s). Suitable pH modifiers are well known in the art. The pH modifier may be an acidic or basic compound. Examples of acidic pH modifiers include, but are not limited to, carboxylic acids, inorganic acids, and sulfonic acids. Examples of basic pH modifiers include, but are not limited to, hydroxides, alkoxides, nitrogen-containing compounds other than primary and secondary amines, and basic carbonates and phosphates.

The aqueous solution(s) or dispersion(s) may optionally include at least one thickener. The thickener may be selected from among known viscosity modifiers, including, but not limited to, polysaccharides and derivatives thereof, such as starch or hydroxyethyl cellulose.

The aqueous solution(s) or dispersion(s) may optionally include at least one antimicrobial agent. Suitable antimicrobial preservatives are well known in the art. Examples of suitable antimicrobials include, but are not limited to, alkyl parabens, such as methylparaben, ethylparaben, propylparaben, and butylparaben; triclosan; chlorhexidine; cresol; chlorocresol; hydroquinone; sodium benzoate; and potassium benzoate.

The aqueous solution(s) or dispersion(s) may also optionally include at least one colorant to enhance the visibility of the solution(s). Suitable colorants include dyes, pigments, and natural coloring agents. Examples of suitable colorants include, but are not limited to, FD&C and D&C colorants, such as FD&C Violet No. 2, FD&C Blue No. 1, D&C Green No. 6, D&C Green No. 5, D&C Violet No. 2; and natural colorants such as beetroot red, canthaxanthin, chlorophyll, eosin, saffron, and carmine.

The aqueous solution(s) or dispersion(s) may also optionally include at least one surfactant. Surfactant, as used herein, refers to a compound that lowers the surface tension of water. The surfactant may be an ionic surfactant, such as sodium lauryl sulfate, or a neutral surfactant, such as polyoxyethylene ethers, polyoxyethylene esters, and polyoxyethylene sorbitan.

Additionally, the aqueous solution(s) or dispersion(s) may optionally include at least one pharmaceutical drug or therapeutic agent. Suitable drugs and therapeutic agents are well known in the art (for example see the United States Pharmacopeia (USP), Physician's Desk Reference (Thomson Publishing), The Merck Manual of Diagnosis and Therapy 18th ed., Mark H. Beers and Robert Berkow (eds.), Merck Publishing Group, 2006; or, in the case of animals, The Merck Veterinary Manual, 9th ed., Kahn, C. A. (ed.), Merck Publishing Group, 2005). Nonlimiting examples include, but are not limited to, anti-inflammatory agents, for example, glucocorticoids such as prednisone, dexamethasone, budesonide; non-steroidal anti-inflammatory agents such as indomethacin, salicylic acid acetate, ibuprofen, sulindac, piroxicam, and naproxen; fibrinolytic agents such as a tissue plasminogen activator and streptokinase; anti-coagulants such as heparin, hirudin, ancrod, dicumarol, sincumar, iloprost, L-arginine, dipyramidole and other platelet function inhibitors; antibodies; nucleic acids; peptides; hormones; growth factors; cytokines; chemokines; clotting factors; endogenous clotting inhibitors; antibacterial agents; antiviral agents; antifungal agents; anti-cancer agents; cell adhesion inhibitors; healing promoters; vaccines; thrombogenic agents, such as thrombin, fibrinogen, homocysteine, and estramustine; radio-opaque compounds, such as barium sulfate and gold particles and radiolabels.

Additionally, the second aqueous solution or dispersion comprising the multi-arm amine may optionally comprise at least one other multi-functional amine having one or more primary amine groups to provide other beneficial properties, such as hydrophobicity or modified crosslink density. The multi-functional amine is capable of inducing gelation when mixed with an oxidized polysaccharide in an aqueous solution or dispersion. Suitable multi-functional amines include, but are not limited to, linear and branched diamines, such as diaminoalkanes, polyaminoalkanes, and spermine; branched polyamines, such as polyethylenimine; cyclic diamines, such as N,N′-bis(3-aminopropyl)piperazine, 5-amino-1,3,3-trimethylcyclohexanemethylamine, 1,3-bis(aminomethyl)cyclohexane, 1,4-diaminocyclohexane, and p-xylylenediamine; aminoalkyltrialkoxysilanes, such as 3-aminopropyltrimethoxysilane and 3-aminopropyltriethoxysilane; aminoalkyldialkoxyalkylsilanes, such as 3-aminopropyldiethoxymethylsilane, dihydrazides, such as adipic dihydrazide; linear polymeric diamines, such as linear polyethylenimine, α,ω-amino-terminated polyethers, α,ω-bis(3-aminopropyl)polybutanediol, β,ω-1-amino-terminated polyethers (linear Jeffamines®); comb polyamines, such as chitosan, polyallylamine, and polylysine, and di- and polyhydrazides, such as bis(carboxyhydrazido)polyethers and poly(carboxyhydrazido) star polyethers. Many of these compounds are commercially available from companies such as Sigma-Aldrich and Huntsman LLC. Typically, if present, the multi-functional amine is used at a concentration of about 5% by weight to about 1000% by weight relative to the weight of the multi-arm amine in the aqueous solution or dispersion.

In another embodiment, the multi-functional amine is provided in a separate third solution at a concentration of about 5% by weight to about 100% by weight relative to the total weight of the solution. If the multi-functional amine is not used neat (i.e., 100% by weight), it is used in the form of an aqueous solution or dispersion. For use on living tissue, it is preferred that the solution comprising the multi-functional amine be sterilized. Any of the methods described above for sterilizing the first and second aqueous solutions or dispersions may be used. The aqueous solution or dispersion comprising the multi-functional amine may further comprise various additives. Any of the additives described above may be used.

The first aqueous solution or dispersion and the second aqueous solution or dispersion may be applied to an anatomical site in the body of humans or animals in any number of ways. The anatomical site may be any external or internal part of the body of humans or animals, including but not limited to, tissues, organs, a fistula, or the punctum (i.e., a tear drainage duct) for the treatment of dry eye syndrome. Once both solutions or dispersions are applied to a site, they crosslink to form a hydrogel, a process referred to herein as curing, typically in about 1 second to about 2 minutes. The first aqueous solution or dispersion and the second aqueous solution or dispersion may be applied to an anatomical site such as a tissue or organ to form a hydrogel coating on the site. Additionally, the two aqueous solutions may be applied to an anatomical site such as a fistula, or the punctum to form a hydrogel which completely or partially fills the site.

In one embodiment, the two aqueous solutions or dispersions are applied to the site sequentially using any suitable means including, but not limited to, spraying, brushing with a cotton swab or brush, or extrusion using a pipette, or a syringe. The solutions or dispersions may be applied in any order. Then, the solutions or dispersions are mixed on the site using any suitable device, such as a cotton swab, a spatula, or the tip of the pipette or syringe.

In another embodiment, the two aqueous solutions or dispersions are premixed manually before application to the site. The resulting mixture is then applied to the site before it completely cures using a suitable applicator, as described above.

In another embodiment, the two aqueous solutions or dispersions are contained in separate barrels of a double-barrel syringe. In this way the two aqueous solutions or dispersions are applied simultaneously to the site with the syringe. Suitable double-barrel syringe applicators are known in the art. For example, Redl describes several suitable applicators for use in the invention in U.S. Pat. No. 6,620,125, (particularly FIGS. 1, 5, and 6, which are described in Columns 4, line 10 through column 6, line 47). Additionally, the double barrel syringe may contain a motionless mixer, such as that available from ConProtec, Inc. (Salem, N.H.) or Mixpac Systems AG (Rotkreuz, Switzerland), at the tip to effect mixing of the two aqueous solutions or dispersions prior to application. Alternatively, the mixing tip may be equipped with a spray head, such as that described by Cruise et al. in U.S. Pat. No.6,458,147. Additionally, the mixture of the two aqueous solutions or dispersions from the double-barrel syringe may be applied to the site using a catheter or endoscope. Devices for mixing a two liquid component tissue adhesive and delivering the resulting mixture endoscopically are known in the art and may be adapted for the mixing and delivery of the two aqueous solutions or dispersions disclosed herein (see for example, Nielson, U.S. Pat. No. 6,723,067; and Redl et al., U.S. Pat. No. 4,631,055). Suitable delivery devices for use in ophthalmic applications, where small volumes of the two aqueous solutions or dispersions or the mixture thereof are required, are also known in the art (see for example Miller et al., U.S. Pat. No. 4,874,368, and copending and commonly owned U.S. Patent Application No. 61/002,071).

In another embodiment, the first aqueous solution or dispersion and the second aqueous solution or dispersion are applied to the site simultaneously where they mix to form a hydrogel. The two aqueous solutions or dispersions may be applied to the site in various ways, for example, using a dual-lumen catheter, such as those available from Bistech, Inc. (Woburn, Mass.). Additionally, injection devices for introducing two liquid components endoscopically into the body simultaneously are known in the art and may be adapted for the delivery of the two aqueous solutions or dispersions disclosed herein (see for example, Linder et al., U.S. Pat. No. 5,322,510).

In another embodiment, the two aqueous solutions or dispersions may be applied to the site using a spray device, such as those described by Fukunaga et al. (U.S. Pat. No. 5,582,596), Delmotte et al. (U.S. Pat. No. 5,989,215) or Sawhney (U.S. Pat. No. 6,179,862).

In another embodiment, the two aqueous solutions or dispersions may be applied to the site using a minimally invasive surgical applicator, such as those described by Sawhney (U.S. Pat. No. 7,347,850).

In another embodiment, the hydrogel sealant of the invention is used to bond at least two anatomical sites together. In this embodiment, the first aqueous solution or dispersion is applied to at least one site, and the second aqueous solution or dispersion is applied to at least one of either the same site or one other site. The two or more sites are contacted and held together manually or using some other means, such as a surgical clamp, for a time sufficient for the mixture to cure, typically from about 1 second to about 2 minutes. Alternatively, a mixture of the two aqueous solutions or dispersions either premixed manually or using a double-barrel syringe applicator, is applied to at least one of the anatomical sites to be bonded. The two or more sites are contacted and held together manually or using some other means, such as a surgical clamp, for a time sufficient for the mixture to cure.

In another embodiment, the low swell, long-lived hydrogel sealant disclosed herein is used in the form of a dried hydrogel. In this embodiment, a hydrogel is prepared by mixing a first solution or dispersion comprising at least one highly oxidized polysaccharide containing aldehyde groups in a first solvent with a second solution or dispersion comprising at least one multi-arm amine in a second solvent, to form the hydrogel. The first solvent may be either the same as or different from the second solvent. If two different solvents are used to prepare the first solution or dispersion and the second solution or dispersion, the two solvents are miscible with each other. Suitable solvents include, but are not limited to, water, ethanol, isopropanol, tetrahydrofuran, hexanes, and polyethylene glycol. In one embodiment, both the first solvent and the second solvent are water.

For the reasons discussed above, where the concentration of the highly oxidized polysaccharide in the first solution or dispersion used to prepare the dried hydrogel is equal to or greater than 6 wt % but less than 8 wt %, the equivalent weight per aldehyde group of the highly oxidized polysaccharide is about 65 to about 70 Daltons in order to obtain a crosslink density that results in a hydrogel with low swell. Where the concentration of the highly oxidized polysaccharide in the first solution or dispersion used to prepare the dried hydrogel is equal to or greater than 8 wt % to about 40 wt %, the equivalent weight per aldehyde group of the highly oxidized polysaccharide is about 65 to about 85 Daltons. The second solution or dispersion used to prepare the dried hydrogel contains the multi-arm amine at a concentration of about of 5% to about 70% by weight, more particularly about 15% to about 50% by weight, and more particularly about 30% to about 50% by weight, relative to the total weight of the solution or dispersion.

The solutions or dispersions used to prepare the dried hydrogel may further comprise various additives depending on the intended application. Any of the additives described above may be used. The hydrogel is then treated to remove at least a portion of the solvent(s) contained therein to form the dried hydrogel. Preferably, substantially all of the solvent(s) is/are removed from the hydrogel. The solvent(s) may be removed from the hydrogel using methods known in the art, for example, using heat, vacuum, a combination of heat and vacuum, or flowing a stream of dry air or a dry inert gas such as nitrogen over the hydrogel. The dried hydrogel may be sterilized using the methods described above.

In one embodiment, the dried hydrogel is used in the form of a film. The dried hydrogel film may be formed by casting a mixture of the first and second solutions or dispersions, as described above, on a suitable substrate and treating the resulting hydrogel to form a dried hydrogel film. The dried hydrogel film may be applied directly to an anatomical site. Additionally, the dried hydrogel film may be used to bond two anatomical sites together.

In another embodiment, the dried hydrogel is used in the form of finely divided particles. The dried hydrogel particles may be formed by comminuting the dried hydrogel using methods known in the art, including, but not limited to, grinding, milling, or crushing with a mortar and pestle. The dried hydrogel particles may be applied to an anatomical site in a variety of ways, such as sprinkling or spraying, and may also be used to bond two anatomical sites together.

### Kits

In one embodiment, the kit comprises at least one highly oxidized polysaccharide having an equivalent weight per aldehyde group of about 65 to about 85 Daltons contained in a first aqueous solution or dispersion at a concentration of 6% to about 40% by weight, and at least one multi-arm amine contained in a second aqueous solution or dispersion, as described above. Each of the aqueous solutions or dispersions may be contained in any suitable vessel, such as a vial or a syringe barrel.

In another embodiment, the kit comprises a dried hydrogel comprising a highly oxidized polysaccharide and a multi-arm amine, prepared as described above. The dried hydrogel may be a film, finely divided particles, or other dried forms. The kit may further comprise a buffer for hydrating the dried hydrogel. The dried hydrogel may be contained in any suitable container.

### Medical Applications

The low swell, long-lived hydrogel sealant disclosed herein may be particularly suitable for applications requiring low swell and slow degradation including, but not limited to, ophthalmic applications such as sealing wounds resulting from trauma such as corneal lacerations, or from surgical procedures such as vitrectomy procedures, cataract surgery, LASIK surgery, glaucoma surgery, and corneal transplants; neurosurgery applications, such as sealing the dura; and as a plug to seal a fistula or the punctum. The low swell, long-lived hydrogel sealant may also be useful as a tissue sealant and adhesive, and as an anti-adhesion barrier. In these applications the first aqueous solution or dispersion and the second aqueous solution or dispersion, or the dried hydrogel may be applied to the desired anatomical site using the methods described above.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations used is as follows: “min” means minute(s), “h” means hour(s), “sec” means second(s), “d” means day(s), “mL” means milliliter(s), “L” means liter(s), “μL” means microliter(s), “cm” means centimeter(s), “mm” means millimeter(s), “μm” means micrometer(s), “mol” means mole(s), “mmol” means millimole(s), “g” means gram(s), “mg” means milligram(s), “kg” means kilogram(s), “wt %” means percent by weight, “mol %” means mole percent, “M” means molar concentration, “Vol” means volume, “v/v” means volume per volume, “Da” means Dalton(s), “kDa” means kiloDalton(s), “mw” means molecular weight, the designation “10K” means that a polymer molecule possesses a weight-average molecular weight of about 10 kiloDaltons, a designation of “60K” indicates a weight-average molecular weight of about 60 kiloDaltons, etc., “MWCO” means molecular weight cut-off, “Pa” means pascal(s), “kPa” means kilopascal(s), “PBS” means phosphate-buffered saline, “PEG” means polyethylene glycol, “rpm” means revolutions per minute.

A reference to “Aldrich” or a reference to “Sigma” means the said chemical or ingredient was obtained from Sigma-Aldrich, St. Louis, Mo.

All water used in these Examples was doubly distilled unless otherwise stated.

### General Methods:

### Preparation of Highly Oxidized Dextrans:

Preparation of the following highly oxidized dextrans, also referred to herein as dextran aldehydes, is described below: (1) D10-73 (the first number is the approximate weight-average molecular weight of the dextran in kDa and the second number is the equivalent weight per aldehyde group of the highly oxidized dextran) having 91% oxidation conversion (equivalent weight per aldehyde group of 73 Daltons) from dextran having a weight-average molecular weight of 8,500 to 11,500 Da, and (2) D60-71 having 93% oxidation conversion (equivalent weight per aldehyde group of 71 Daltons) from dextran having a weight-average molecular weight of 60,000 to 90,000 Da. Oxidized dextrans of the same average molecular weights having other oxidation conversions were prepared by varying the amount of the sodium periodate used. For example, the following dextran aldehydes were also prepared: weight-average molecular weight of 8,500-11,500 Da with an oxidation conversion of 50%, equivalent weight per aldehyde group of about 146 Da (D10-146); weight-average molecular weight of 8,500-11,500 Da with an oxidation conversion of 84%, equivalent weight per aldehyde group of about 80 Da (D10-80); weight-average molecular weight of 8,500-11,500 Da with an oxidation conversion of 76%, equivalent weight per aldehyde group of about 91 Da (D10-91); and weight-average molecular weight of 60,000-90,000 Da with an oxidation conversion of 50%, equivalent weight per aldehyde group of about 146 Da (D60-146).

Preparation of Oxidized Dextran D10-73:

In a round bottom flask, 20 g of dextran (weight-average molecular weight of 8,500 to 11,500 Daltons, Sigma D9260) was dissolved in 180 mL of water. Sodium periodate solution (41.1 g in 360 mL of water) was added to the round bottom flask. The reaction mixture was stirred at room temperature for 5 h, and then dialyzed in water (MWCO 3500 dialysis membrane tube, 6 water exchanges). The dialysates were combined, and freeze dried to yield 13.1 g of white solid.

The oxidation conversion was determined by ¹H NMR from the ratio (R) of the integral of the O₂CH— region (from 6.3 ppm to 4.2 ppm minus the water peak) versus the ratio of the —OCH-region (from 4.2 ppm to 3 ppm). The calculation is as follows:

Degree of unoxidized glucose units (X)=(300R−300)/(3+2R)

% of oxidation=(100−100X)

Using this method, the oxidation conversion was found to be 91%, equivalent weight per aldehyde group of about 73 Daltons. The oxidized dextran was analyzed by size exclusion chromatography: M_{w}=9.9×10³, M_{w}/Mₙ=1.9.

Preparation of Oxidized Dextran D60-71:

In a round bottom flask, 15 g of dextran (weight-average molecular weight of 60,000 to 90,000 Daltons, Sigma D3759) was dissolved in 135 mL of water. Sodium periodate solution (33 g in 200 mL of water) was added to the round bottom flask. The reaction mixture was stirred at room temperature for 5 h, and then dialyzed in water (MWCO 3500 dialysis membrane tube, 6 water exchanges). The dialysates were combined, and freeze dried to yield 15.6 g of white solid.

The oxidation conversion was determined by ¹H NMR to be 93%, as described above, equivalent weight per aldehyde group of about 71 Daltons.

### Preparation of Multi-Arm PEG Amines:

Preparation of 8-Arm Polyethylene Glycol 10K Octaamine (P8-10-1):

An 8-arm PEG 10K octaamine, referred to herein as “P8-10-1,” was synthesized using the two-step procedure described by Chenault in co-pending and commonly owned U.S. Patent Application Publication No. 2007/0249870. A typical synthesis is described here. In the first step, the 8-arm PEG 10K is converted to an 8-Arm PEG 10K chloride by reaction with thionyl chloride, i.e.,

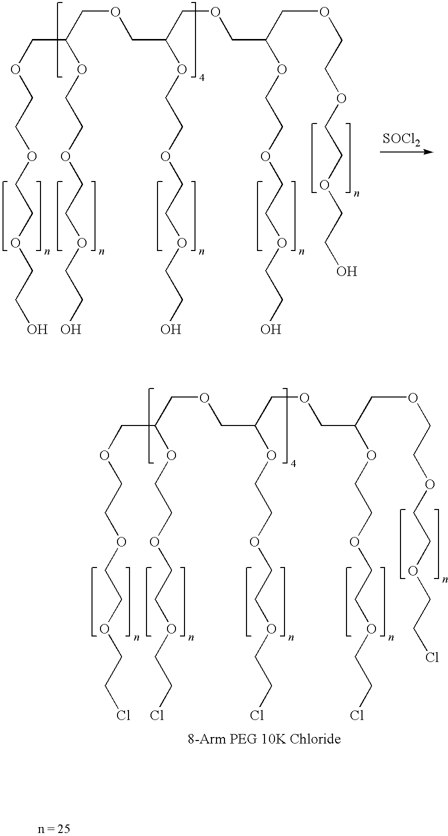

The 8-arm PEG 10K (NOF Sunbright HGEO-10000, NOF Corp., Tokyo, Japan; 1000 g in a 3-L round-bottom flask) is dried by dissolving it in 1.5 L of toluene and distilling 500 mL of toluene-water azeotrope plus toluene under reduced pressure (2 kPa) with a pot temperature of 60° C., adding another 500 mL of toluene to the pot, and distilling 500 mL of toluene-water azeotrope plus toluene under reduced pressure (2 kPa) with a pot temperature of 60° C.

The solution of 8-arm PEG is allowed to cool to room temperature. Then, thionyl chloride (233 mL, 3.19 mol) is added to the flask, which is equipped with a reflux condenser, and the mixture is heated at 85° C. with stirring under a blanket of nitrogen for 4 h. Excess thionyl chloride and most of the toluene are removed by vacuum distillation at 2 kPa (bath temp 40-60° C.). Two successive 500-mL portions of toluene are added and evaporated under reduced pressure (2 kPa, bath temperature 80-85° C.) to complete the removal of thionyl chloride. The final crude product is dissolved in 1000 g of de-ionized water.

In the second step, the 8-Arm PEG 10K chloride is converted to the 8-Arm PEG 10K amine by reaction with aqueous ammonia, i.e.,

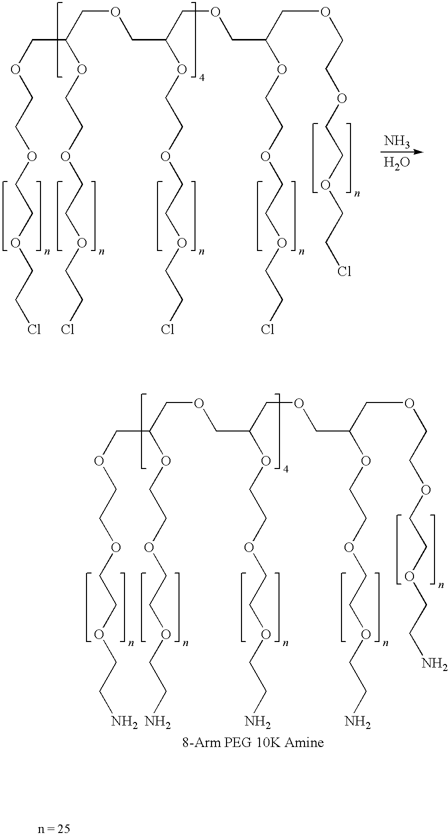

The aqueous solution of 8-arm PEG-Cl prepared above, is dissolved in 16 L of concentrated aqueous ammonia (28 wt %) and heated in a sealed stainless steel pressure vessel at 60° C. for 48 h. The solution is sparged for 24 h with dry nitrogen and then placed under reduced pressure for 3 h to drive off ammonia. The solution is then passed through a column of strongly basic anion exchange resin (5 kg; Purolite® A-860, The Purolite Co., Bala-Cynwyd, Pa.) in the hydroxide form. The eluant is collected, and two 7-L portions of de-ionized water are passed through the column and collected. The aqueous fractions are combined, concentrated under reduced pressure (2 to 0.3 kPa, bath temperature 60° C.) to give the 8-Arm PEG 10K octaamine (P8-10-1). The final product is characterized by proton NMR and size exclusion chromatography (SEC), as described by Chenault, supra.

### Examples 1-10

### In Vitro Degradation of Hydrogels—Comparison at the Same Solids Content

The purpose of these Examples was to demonstrate that the hydrogels formed by reaction of a highly oxidized dextran aldehyde with a multi-arm PEG amine degrade in vitro much more slowly than a hydrogel formed from a less oxidized dextran aldehyde and a multi-arm PEG amine, at the same solids content.

The hydrogel samples were prepared by mixing equal volumes of an aqueous solution of a dextran aldehyde and an aqueous solution of a multi-arm PEG amine, as shown in Table 1. After the hydrogels cured, the samples were weighed and placed inside jars containing PBS (phosphate buffered saline) at pH 7.4. The jars were placed inside a temperature-controlled shaker set at 80 rpm and 37° C. The samples were removed from the jars at various times, blotted to remove excess solution, and weighed. Then, the samples were returned to the jars.

The results are summarized in Table 1. The degradation day is defined as the day when the gel was dissolved. The maximum swell reported in the table is the maximum weight of the hydrogel measured during the course of the study divided by the initial weight of the hydrogel, multiplied by 100.

TABLE 1
Results of In Vitro Degradation of Hydrogels

| | Dextran | | | |
|---|---|---|---|---|
| | Aldehyde | PEG Amine | Degradation | Maximum |
| Example | Solution | Solution | (days) | Swell (%) |
| 1 | D10-73 | P8-10-1 | >22 | 314 |
| | 8 wt % | 50 wt % | | |
| 2 | D10-80 | P8-10-1 | >22 | 360 |
| | 8 wt % | 50 wt % | | |
| 3 | D10-146 | P8-10-1 | 8 | 382 |
| Comparative | 8 wt % | 50 wt % | | |
| 4, | D10-91 | P8-10-1 | >22 | 410 |
| Comparative | 8 wt % | 50 wt % | | |
| 5 | D10-73 | P8-10-1 | >22 | 138 |
| | 8 wt % | 30 wt % | | |
| 6 | D10-80 | P8-10-1 | >22 | 167 |
| | 8 wt % | 30 wt % | | |
| 7 | D10-146 | P8-10-1 | 5 | 400 |
| Comparative | 8 wt % | 30 wt % | | |
| 8, | D10-91 | P8-10-1 | >22 | 221 |
| Comparative | 8 wt % | 30 wt % | | |
| 9 | D60-71 | P8-10-1 | >22 | 167 |
| | 8 wt % | 30 wt % | | |
| 10 | D60-146 | P8-10-1 | 2 | 309 |
| Comparative | 8 wt % | 30 wt % | | |

As can be seen from the data in Table 1, hydrogels formed from highly oxidized dextran aldehydes (i.e., Examples 1, 2, 5, 6 and 9) had longer degradation times and lower maximum swell than hydrogels formed from dextran aldehyde having a lower oxidation level (i.e., Comparative Examples 3, 7, and 10) at the same solids content. Hydrogels formed from oxidized dextran having an equivalent weight per aldehyde group of 91 Daltons (Comparative Examples 4 and 8) had a degradation time comparable to that of the hydrogels formed from the highly oxidized dextrans, but had a higher maximum swell.

### Examples 11 and 12

### In Vitro Biocompatibility Testing—Cytotoxicity

The purpose of these Examples was to demonstrate the safety of hydrogels resulting from the reaction of a multi-arm PEG amine with a highly oxidized dextran aldehyde in an in vitro test.

The testing was done using NIH3T3 human fibroblast cell cultures according to ISO10993-5:1999. The NIH3T3 human fibroblast cells were obtained from the American Type Culture Collection (ATCC; Manassas, Va.) and were grown in Dulbecco's modified essential medium (DMEM), supplemented with 10% fetal calf serum.

NIH3T3 human fibroblast cell cultures were challenged with hydrogels made by combining equal volumes of an aqueous solution of a highly oxidized-dextran aldehyde and an aqueous solution of a multi-arm PEG amine, as shown in Table 2. Each hydrogel was placed in a well in a polystyrene culture plate such that about ¼ of the well bottoms were covered. The wells were then sterilized under UV light and seeded with 50,000-100,000 NIH3T3 cells.

The cells grew normally confluent and coated the well bottom, growing up to the edges of the hydrogels; however, they did not overgrow the hydrogels. These results, summarized in Table 2, demonstrate a lack of cytotoxicity of the hydrogels, as well as the lack of adhesion of cell cultures to the hydrogels.

TABLE 2
Cytotoxicity Results

| | | Multi-Arm | |
|---|---|---|---|
| | Dextran Aldehyde | PEG | |
| Example | Solution | Amine Solution | Cytotoxicity |
| 11 | D10-73 | P8-10-1 | nontoxic |
| | 8 wt % | 50 wt % | |
| 12 | D60-71 | P8-10-1 | nontoxic |
| | 8 wt % | 50 wt % | |

### Examples 13 and 14 (Comparative)

### In-Vitro Burst Testing of a Sealed Scalpel Incision in Pig Intestine

The purpose of these Examples was to demonstrate the burst strength of a seal made with various hydrogels of an incision made in the small intestine from a pig.

A syringe pump system was used to measure the burst strength of a seal of an incision made in a section of small intestine from a pig. The syringe pump (Model No. 22, Harvard Apparatus, Holliston, Mass.) was modified to be equipped with two 30 mL syringes, which were connected together through a “Y” junction. Water was pumped through a single piece of Tygon® R-36 tubing (0.6 cm diameter) and through a pressure gauge (Model PDG 5000L, Omega Engineering, Stamford, Conn.).

An approximately 12.5 cm section of clean pig intestine, obtained from a local abattoir, was fitted on one end with a metal plug with a feed line fitting for water feed from the syringe pump and on the other end with a metal plug with a threaded hole which could be sealed with a machine screw. The plugs were held in place with nylon ties around the outside of the intestine. An incision was made through the intestine wall into the interior by puncturing with a Bard Parker™ surgical blade handle 5 (obtained from BD Surgical Products, Franklin Lakes, N.J.), fitted with a #15 surgical blade. The incision on the outside of the intestine was wider than the scalpel blade (typically 4-5 mm) while the hole through the inside wall was about 3 mm (about equal to the blade). This size incision mimics the distance between the interrupted sutures if an intestine were to be cut and later sutured. The intestine was filled with water containing a purple dye via the syringe pump until water began to leak from the open hole in the end plug and also from the scalpel puncture in the intestinal wall. The pump was then turned off and the end plug was sealed with the machine screw. The scalpel incision site was blotted dry using a paper towel.

The dextran aldehyde and multi-arm PEG amine solutions were prepared in water. The two solutions were applied to the incision using a double barrel syringe (Mixpac Systems AG (Rotkreuz, Switzerland) fitted with a 16 step static mixer (Mixpac Systems AG). After the application, the adhesive was allowed to cure at room temperature for no longer than 2 min.

Burst pressure testing, also referred to herein as leak pressure testing, was done by pressurizing the sealed intestine with water from the syringe pump at a flow rate of 11 mL/min until the bioadhesive seal began to leak, at which point the pressure was recorded. Adhesive failure was attributed when the water leaked under the seal between the hydrogel and the tissue surface. Cohesive failure was attributed when the water penetrated and leaked through the hydrogel itself. Burst pressure testing was also done on the unsealed intestine and the leak pressure was less than 10 mm of mercury (Hg) (less than 1.3 kPa). The results of the burst testing are summarized in Table 3. The results demonstrate that the hydrogel formed by reaction of a highly oxidized dextran aldehyde and multi-arm PEG amine solutions (i.e., Example 13) was able to seal the incision in the pig intestine and gave higher burst pressure than the hydrogel prepared from oxidized dextran aldehyde having a lower oxidation level and a multi-arm PEG amine (i.e., Comparative Example 14) at the same solids content.

TABLE 3
Burst Pressure Testing Results

| | | | | Standard |
|---|---|---|---|---|
| | | Multi-Arm | | Deviation |
| | Dextran | PEG Amine | Ave Burst | Burst |
| | Aldehyde | Solution | Pressure, | Pressure, |
| Example | Solution | (P8-10-1) | mm Hg | mm Hg |
| 13 | D10-73 | 50 wt % | 116.1 | 23.8 |
| | 8 wt % | | (15.5 kPa) | (3.2 kPa) |
| 14, | D10-146 | 50 wt % | 89.1 | 20.0 |
| Comparative | 8 wt % | | (11.9 kPa) | (2.7 kPa) |

The results demonstrate that the hydrogel formed by reaction of highly oxidized dextran aldehyde and multi-arm PEG amine solutions (Example 13) was able to seal the incision in the pig intestine and gave higher burst pressure than hydrogel prepared from oxidized dextran aldehyde having a lower oxidation level and a multi-arm PEG amine (Comparative Example 14) at the same solids content.

### Examples 15 and 16 (Comparative)

### Tensile Strength Testing

The purpose of these Examples was to demonstrate the tensile strength of hydrogels formed by reaction of a highly oxidized dextran aldehyde with a multi-arm PEG amine.

The hydrogel samples were prepared by mixing equal volumes of an aqueous solution of a dextran aldehyde and an aqueous solution of a multi-arm PEG amine, as shown in Table 4. Tensile strength testing was done using the following method.

A 1:1 v/v double-barrel syringe (Mixpac Systems AG, Rotkreuz, Switzerland) was loaded with the two reactive solutions, dextran aldehyde solution in one side and a multi-arm PEG amine solution in the other. The syringe was fitted with a 2.5-mm diameter mixing tip having 16 static mixing elements (Mixpac No. 2.5-16-DM) to dispense sealant. Two 1 inch×3 inch (2.5 cm×7.5 cm) microscope slides were laid parallel to one another, exactly 0.68 cm apart using a spacer, on a sheet of silicone rubber which had been lightly rubbed with silicone stopcock grease as a release agent. A bead of mixed sealant solution was quickly delivered onto the silicone rubber surface between the two slides and promptly covered with a 5 cm×7.5 cm microscope slide to compress the still-fluid sealant to a flat strip between the two slides. The strip was allowed to cure for 15 min; then the silicone sheet was carefully peeled away, leaving the hydrogel strip on the glass slide. The two 1 inch×3 inch (2.5 cm×7.5 cm) slides were carefully removed and finally the adhering hydrogel strip was carefully peeled off the 5 cm×7.5 cm microscope slide. Sample strips were tested immediately after molding. If there were no flaws or bubbles, the strip could be cut in the center to give two test pieces, each approximately 3 cm long.

Tensile strength was determined using an Exceed Texture Analyzer (Stable Microsystems, Surrey, England) with clamps for pulling films. Pieces of smooth silicone rubber sheet were taped with double-sided tape onto the clamp faces to lightly grip the hydrogels without squashing them. The hydrogel strips were clamped with a gauge length of 1.00 cm (about 1 cm of each end of the 3-cm hydrogel strip was in each clamp) and pulled at a rate of 1 cm/min for 10 cm or until break. After break, the thickness of the hydrogel strip at the break point was measured with a micrometer to calculate tensile strength. The results are summarized in Table 4.

TABLE 4
Results of Tensile Strength Testing

| | Dextran | | |
|---|---|---|---|
| | Aldehyde | PEG Amine | Tensile Strength |
| Example | Solution | Solution (P8-10-1) | (g/cm²) |
| 15 | D10-73 | 50 wt % | 2824 |
| | 8 wt % | | |
| 16, | D10-146 | 50 wt % | 1649 |
| Comparative | 8 wt % | | |

The results demonstrate that hydrogel comprised of highly oxidized dextran aldehyde (Example 15) has greater tensile strength than the hydrogel formed using dextran aldehyde having a lower oxidization level (Comparative Example 16) at the same solids content.

What is claimed is:

1. A kit comprising:
a) a first aqueous solution or dispersion comprising at least one highly oxidized polysaccharide containing aldehyde groups, said highly oxidized polysaccharide having a weight-average molecular weight of about 1,000 to about 1,000,000 Daltons and an equivalent weight per aldehyde group of about 65 to about 85 Daltons, wherein said first aqueous solution or dispersion contains said highly oxidized polysaccharide at a concentration of 6% to about 40% by weight; and
b) a second aqueous solution or dispersion comprising at least one water-dispersible, multi-arm amine wherein at least three of the arms are terminated by at least one primary amine group, said multi-arm amine having a number-average molecular weight of about 450 to about 200,000 Daltons, wherein said second aqueous solution or dispersion contains said multi-arm amine at a concentration of about 5% to about 70% by weight;
provided that:
(i) if the concentration of the highly oxidized polysaccharide in the first aqueous solution or dispersion is equal to or greater than 6 wt % but less than 8 wt %, the equivalent weight per aldehyde group of the highly oxidized polysaccharide is about 65 to about 70 Daltons;
(ii) if the concentration of the highly oxidized polysaccharide in the first aqueous solution or dispersion is equal to or greater than 8 wt % to about 40 wt %, the equivalent weight per aldehyde group of the highly oxidized polysaccharide is about 65 to about 85 Daltons.

2. The kit according to claim 1 wherein the polysaccharide is selected from the group consisting of dextran, starch, agar, cellulose, and hyaluronic acid.

3. The kit according to claim 1 wherein the water-dispersible, multi-arm amine is selected from the group consisting of amino-terminated star polyethylene oxides, amino-terminated dendritic polyethylene oxides, amino-terminated comb polyethylene oxides, amino-terminated star polypropylene oxides, amino-terminated dendritic polypropylene oxides, amino-terminated comb polypropylene oxides, amino-terminated star polyethylene oxide-polypropylene oxide copolymers, amino-terminated dendritic polyethylene oxide-polypropylene oxide copolymers, amino-terminated comb polyethylene oxide-polypropylene oxide copolymers, polyoxyalkylene triamines, amino-terminated dendritic polyamidoamines, and multi-arm branched end amines.

4. The kit according to claim 1 wherein the polysaccharide is dextran and the water-dispersible, multi-arm amine is a multi-arm polyethylene glycol amine.

5. The kit according to claim 1 further comprising at least one multi-functional amine having one or more primary amine groups, wherein said multi-functional amine is contained in at least one of the following:
a) the first aqueous solution or dispersion;
b) the second aqueous solution or dispersion;
c) a third solution.

6. A dried hydrogel formed by a process comprising the steps of:
a) combining (i) a first solution or dispersion comprising at least one highly oxidized polysaccharide containing aldehyde groups in a first solvent, said oxidized polysaccharide having a weight-average molecular weight of about 1,000 to about 1,000,000 Daltons and an equivalent weight per aldehyde group of about 65 to about 85 Daltons, wherein said first solution or dispersion contains said highly oxidized polysaccharide at a concentration of 6% to about 40% by weight; with (ii) a second solution or dispersion comprising at least one water-dispersible, multi-arm amine in a second solvent, wherein at least three of the arms of said multi-arm amine are terminated by at least one primary amine group, said multi-arm amine having a number-average molecular weight of about 450 to about 200,000 Daltons, wherein said second solution or dispersion contains said multi-arm amine at a concentration of about 5% to about 70% by weight, to form a hydrogel, wherein the first solvent is either the same as or different from the second solvent; and
b) treating the hydrogel to remove at least a portion of said first solvent and said second solvent to form the dried hydrogel;
provided that:
(i) if the concentration of the highly oxidized polysaccharide in the first solution or dispersion is equal to or greater than 6 wt % but less than 8 wt %, the equivalent weight per aldehyde group of the highly oxidized polysaccharide is about 65 to about 70 Daltons;
(ii) if the concentration of the highly oxidized polysaccharide in the first solution or dispersion is equal to or greater than 8 wt % to about 40 wt %, the equivalent weight per aldehyde group of the highly oxidized polysaccharide is about 65 to about 85 Daltons.

7. The dried hydrogel according to claim 6 wherein said dried hydrogel is in the form of a film.

8. The dried hydrogel according to claim 6 wherein the process further comprises the step of comminuting the dried hydrogel to form finely divided particles.

9. The dried hydrogel according to claim 6 wherein the polysaccharide is selected from the group consisting of dextran, starch, agar, cellulose, and hyaluronic acid.

10. The dried hydrogel according to claim 6 wherein the water-dispersible, multi-arm amine is selected from the group consisting of amino-terminated star polyethylene oxides, amino-terminated dendritic polyethylene oxides, amino-terminated comb polyethylene oxides, amino-terminated star polypropylene oxides, amino-terminated dendritic polypropylene oxides, amino-terminated comb polypropylene oxides, amino-terminated star polyethylene oxide-polypropylene oxide copolymers, amino-terminated dendritic polyethylene oxide-polypropylene oxide copolymers, amino-terminated comb polyethylene oxide-polypropylene oxide copolymers, polyoxyalkylene triamines, amino-terminated dendritic polyamidoamines, and multi-arm branched end amines.

11. The dried hydrogel according to claim 6 wherein the polysaccharide is dextran and the multi-arm amine is a multi-arm polyethylene glycol amine.

12. The dried hydrogel according to claim 6 wherein the first solvent and the second solvent are water.

13. A method for applying a coating to an anatomical site on tissue of a living organism comprising: applying to the site (a) a first aqueous solution or dispersion comprising at least one highly oxidized polysaccharide containing aldehyde groups, said highly oxidized polysaccharide having a weight-average molecular weight of about 1,000 to about 1,000,000 Daltons and an equivalent weight per aldehyde group of about 65 to about 85 Daltons, wherein said first aqueous solution or dispersion contains said highly oxidized polysaccharide at a concentration of 6% to about 40% by weight; followed by (b) a second aqueous solution or dispersion comprising at least one water-dispersible, multi-arm amine wherein at least three of the arms are terminated by at least one primary amine group, said multi-arm amine having a number-average molecular weight of about 450 to about 200,000 Daltons, wherein said second aqueous solution or dispersion contains said multi-arm amine at a concentration of about 5% to about 70% by weight, or
applying (b) followed by (a) and mixing (a) and (b) on the site, or
premixing (a) and (b) to form a mixture and applying said mixture to the site;
provided that:
(i) if the concentration of the highly oxidized polysaccharide in the first aqueous solution or dispersion is equal to or greater than 6 wt % but less than 8 wt %, the equivalent weight per aldehyde group of the highly oxidized polysaccharide is about 65 to about 70 Daltons;
(ii) if the concentration of the highly oxidized polysaccharide in the first aqueous solution or dispersion is equal to or greater than 8 wt % to about 40 wt %, the equivalent weight per aldehyde group of the highly oxidized polysaccharide is about 65 to about 85 Daltons.

14. The method according to claim 13 wherein the polysaccharide is dextran and the multi-arm amine is a multi-arm polyethylene glycol amine.

15. A composition comprising the reaction product of:
a) a first aqueous solution or dispersion comprising at least one highly oxidized polysaccharide containing aldehyde groups, said highly oxidized polysaccharide having a weight-average molecular weight of about 1,000 to about 1,000,000 Daltons and an equivalent weight per aldehyde group of about 65 to about 85 Daltons, wherein said first aqueous solution or dispersion contains said highly oxidized polysaccharide at a concentration of 6% to about 40% by weight; and
b) a second aqueous solution or dispersion comprising at least one water-dispersible, multi-arm amine wherein at least three of the arms are terminated by at least one primary amine group, said multi-arm amine having a number-average molecular weight of about 450 to about 200,000 Daltons, wherein said second aqueous solution or dispersion contains said multi-arm amine at a concentration of about 5% to about 70% by weight;
provided that:
(i) if the concentration of the highly oxidized polysaccharide in the first aqueous solution or dispersion is equal to or greater than 6 wt % but less than 8 wt %, the equivalent weight per aldehyde group of the highly oxidized polysaccharide is about 65 to about 70 Daltons;
(ii) if the concentration of the highly oxidized polysaccharide in the first aqueous solution or dispersion is equal to or greater than 8 wt % to about 40 wt %, the equivalent weight per aldehyde group of the highly oxidized polysaccharide is about 65 to about 85 Daltons.

16. The composition according to claim 15 wherein the polysaccharide is selected from the group consisting of dextran, starch, agar, cellulose, and hyaluronic acid.

17. The composition according to claim 15 wherein the water-dispersible multi-arm amine is selected from the group consisting of amino-terminated star polyethylene oxides, amino-terminated dendritic polyethylene oxides, amino-terminated comb polyethylene oxides, amino-terminated star polypropylene oxides, amino-terminated dendritic polypropylene oxides, amino-terminated comb polypropylene oxides, amino-terminated star polyethylene oxide-polypropylene oxide copolymers, amino-terminated dendritic polyethylene oxide-polypropylene oxide copolymers, amino-terminated comb polyethylene oxide-polypropylene oxide copolymers, polyoxyalkylene triamines, amino-terminated dendritic polyamidoamines, and multi-arm branched end amines.

18. The composition according to claim 15 wherein the polysaccharide is dextran and the water-dispersible, multi-arm amine is a multi-arm polyethylene glycol amine.
